(19) United States
(12) Patent Application Publication
(10) Pub. No.: US 2005/0245975 A1
(43) Pub. Date: Nov. 3, 2005

(54) Method and apparatus for controlling delivery of pacing pulses in response to increased ectopic frequency

(76) Inventors: Douglas A. Hettrick, Blaine, MN (US); Paul D. Ziegler, Minneapolis, MN (US); Jacqueline Mittelstadt, Roseville, IL (US); Rahul Mehra, Stillwater, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/824,789

(22) Filed: Apr. 15, 2004

(51) Int. Cl. A61N 1/362

(52) U.S. Cl. 607/9

(57) ABSTRACT

A method and device for controlling delivery of therapy in an implantable device that includes sensing a plurality of events, detecting whether there is an increase in the frequency of first events of the plurality of events corresponding to onset of a second event of the plurality of sensed events, adjusting parameters associated with delivery of the therapy in response to the detected increased frequency of first sensed events, and delivering the therapy using the adjusted parameters.

### FIELD OF THE INVENTION

The present invention relates generally to implantable medical devices, and in particular, the present invention relates to implantable medical devices that deliver pacing pulses in response to a premature contraction to prevent onset of atrial tachyarrhyhmias.

### BACKGROUND OF THE INVENTION

Tachyarrhythmias are episodes of high-rate cardiac depolarizations, which may occur in one chamber of the heart or may be propagated from one chamber to another. Some tachyarrhythmias are sufficiently high in rate to compromise cardiac output from the chamber affected, leading to loss of consciousness or death in the case of ventricular fibrillation, or weakness and dizziness in the case of atrial fibrillation. Atrial tachyarrhythmia is often debilitating, due to the loss of atrial cardiac output, and may sometimes lead to ventricular fibrillation.

Fibrillation may be terminated by administering high energy level cardioversion or defibrillation shocks until the fibrillation is terminated. For example, an implanted device may deliver defibrillation shocks via an electrode carried by a lead implanted within the heart. Unfortunately, the high energy levels associated with cardioversion/defibrillation shocks can cause significant pain to the patient. In addition, atrial defibrillation shocks can sometimes give rise to ventricular arrhythmias. Therefore, it is generally desirable to avoid the onset of atrial tachyarrhythmia, and the need to apply defibrillation shocks.

Some implanted devices deliver anti-tachycardia pacing pulses to terminate detected episodes of atrial tachycardia. Other devices are configured to deliver pacing pulses to prevent the atrial tachyarrhythmia from occurring. In particular, a device may be configured to detect premature atrial contractions (PACs) as trigger events that may indicate the onset of atrial tachyarrhythmia. Delivery of pacing pulses in response to PAC detection can help prevent or decrease the occurrence of atrial tachyarrhythmia. Pacing pulses delivered in response to PAC detection are sometimes referred to as post-PAC pacing pulses.

Atrial tachyarrhythmia occurs when a trigger, such as a sudden change in the electrophysiologic, autonomic, ischemic or mechanical state of the atrium, occurs in a substrate capable of sustaining the arrhythmia. The number and coupling interval of PACs required to initiate atrial tachyarrhythmia is dependent upon the substrate. In a very diseased substrate, for example, a single PAC at a long coupling interval may be sufficient, whereas in patients with more normal substrate, multiple closely coupled premature beats are needed to initiate atrial tachyarrhythmia. For a unique substrate, the number of beats required to initiate atrial tachyarrhythmia is dependent upon their coupling interval (i.e., the shorter the coupling, the fewer number of PACs required).

The inventors have found that initiation of atrial tachyarrhythmias is often preceded by a burst of shortly coupled premature beats that is sometimes, but not always, preceded by isolated premature beats arising from the same site, and the frequency of which tend to increase in the few moments prior to the onset of AF. For example, the frequency of these PACs may increase from 0.8 PACs per minute to 6.2 PACs per minute in the 30 seconds prior to onset of AF. Accordingly, what is needed is a method and apparatus for controlling delivery of pacing pulses in response to changes in the frequency or coupling interval of PACs.

### BRIEF DESCRIPTION OF THE DRAWINGS

Aspects and features of the present invention will be readily appreciated as the same becomes better understood by reference to the following detailed description when considered in connection with the accompanying drawings, wherein:

FIG. 1 is a schematic diagram of an implantable medical device of a type in which the present invention may usefully be practiced;

FIG. 2 is a functional schematic diagram of an implantable medical device of the type illustrated in FIG. 1, in which the present invention may usefully be practiced;

FIG. 3 is a flowchart of generation of a template for an implantable medical device according to an embodiment of the present invention;

FIG. 4 is a flowchart of generation of a template for an implantable medical device according to an embodiment of the present invention;

FIG. 5 is a flowchart of generation of a template for an implantable medical device according to an alternate embodiment of the present invention; and

FIG. 6 is a flowchart of validation of a template for an implantable medical device according to an embodiment of the present invention.

### DETAILED DESCRIPTION OF THE INVENTION

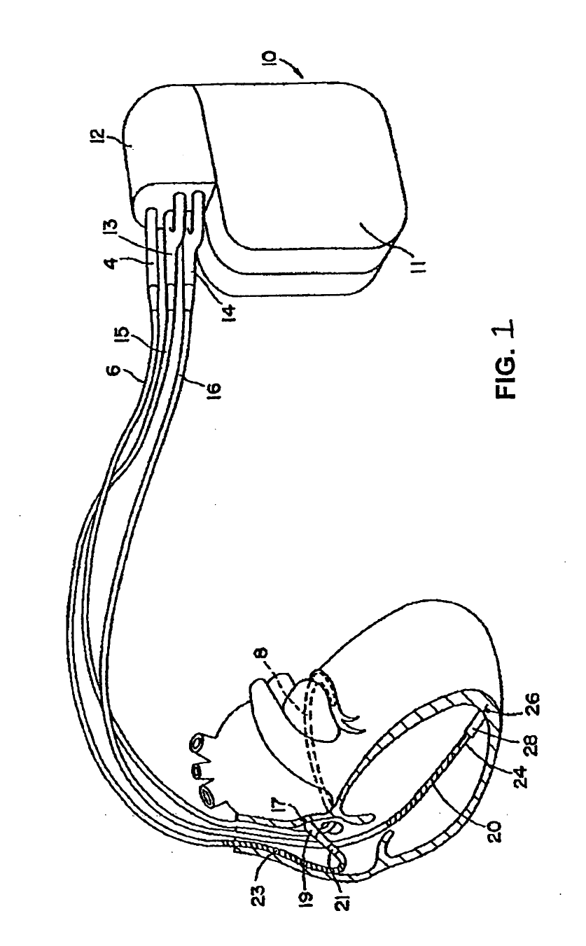

FIG. 1 is a schematic diagram of an implantable medical device of a type in which the present invention may usefully be practiced. As illustrated in FIG. 1, an implantable medical device 10, such as an implantable cardioverter defibrillator (ICD), for example, is coupled to a heart of a patient by way of one or more leads 6, 15, and 16. A connector block 12 receives the proximal end of a right ventricular lead 16, a right atrial lead 15 and a coronary sinus lead 6, used for positioning electrodes for sensing and stimulation in three or four heart chambers. In FIG. 1, right ventricular lead 16 is positioned such that its distal end is in the right ventricle for sensing right ventricular cardiac signals and delivering pacing or shocking pulses in the right ventricle. For these purposes, right ventricular lead 16 is equipped with a ring electrode 24, an extendable helix electrode 26 mounted retractably within an electrode head 28, and a coil electrode 20, each of which are connected to an insulated conductor within the body of lead 16. The proximal end of the insulated conductors are coupled to corresponding connectors carried by bifurcated connector 14 at the proximal end of lead 16 for providing electrical connection to the device 10.

The right atrial lead 15 is positioned such that its distal end is in the vicinity of the right atrium and the superior vena cava. Lead 15 is equipped with a ring electrode 21 and an extendable helix electrode 17, mounted retractably within electrode head 19, for sensing and pacing in the right atrium. Lead 15 is further equipped with a coil electrode 23 for delivering high-energy shock therapy. The ring electrode 21, the helix electrode 17 and the coil electrode 23 are each connected to an insulated conductor with the body of the right atrial lead 15. Each insulated conductor is coupled at its proximal end to a connector carried by bifurcated connector 13.

The coronary sinus lead 6 is advanced within the vasculature of the left side of the heart via the coronary sinus and great cardiac vein. The coronary sinus lead 6 is shown in the embodiment of FIG. 1 as having a defibrillation coil electrode 8 that may be used in combination with either the coil electrode 20 or the coil electrode 23 for delivering electrical shocks for cardioversion and defibrillation therapies. In other embodiments, coronary sinus lead 6 may also be equipped with a distal tip electrode and ring electrode for pacing and sensing functions in the left chambers of the heart. The coil electrode 8 is coupled to an insulated conductor within the body of lead 6, which provides connection to the proximal connector 4.

The electrodes 17 and 21 or 24 and 26 may be used as true bipolar pairs, commonly referred to as a “tip-to-ring” configuration. Further, electrode 17 and coil electrode 20 or electrode 24 and coil electrode 23 may be used as integrated bipolar pairs, commonly referred to as a “tip-to-coil” configuration. In some cases, electrodes 17, 21, 24, and 26 may be used individually in a unipolar configuration with the device housing 11 serving as the indifferent electrode, commonly referred to as the “can” or “case” electrode.

The device housing 11 may also serve as a subcutaneous defibrillation electrode in combination with one or more of the defibrillation coil electrodes 8, 20 or 23 for defibrillation of the atria or ventricles. It is recognized that alternate lead systems may be substituted for the three lead system illustrated in FIG. 1. While a particular multi-chamber ICD and lead system is illustrated in FIG. 1, methodologies included in the present invention may adapted for use with any single chamber, dual chamber, or multi-chamber ICD or pacemaker system, or other cardiac monitoring device.

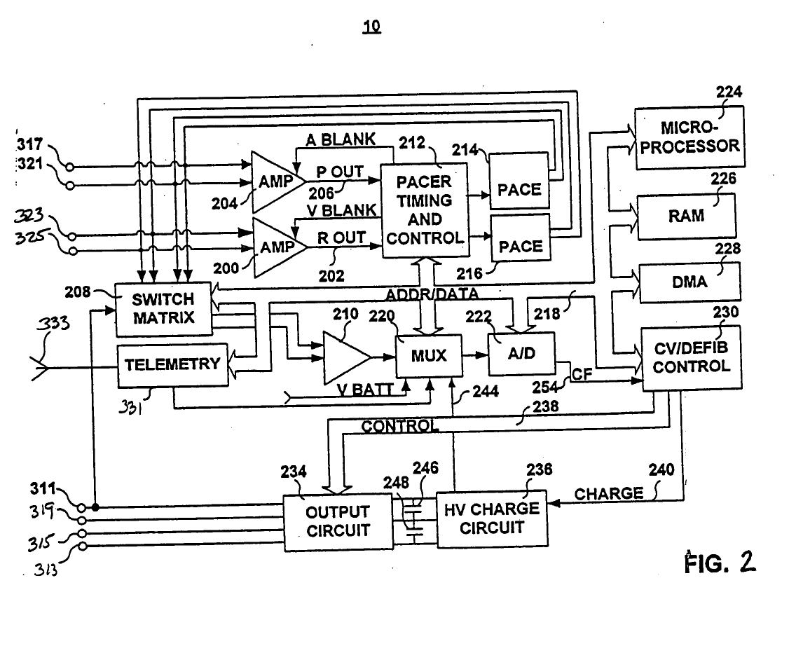

FIG. 2 is a functional schematic diagram of an implantable medical device of the type illustrated in FIG. 1, in which the present invention may usefully be practiced. This diagram should be taken as exemplary of the type of device with which the invention may be embodied and not as limiting. The disclosed embodiment shown in FIG. 2 is a microprocessor-controlled device, but the methods of the present invention may also be practiced with other types of devices such as those employing dedicated digital circuitry.

With regard to the electrode system illustrated in FIG. 1, device 10 is provided with a number of connection terminals for achieving electrical connection to the leads 6, 15, and 16 and their respective electrodes. A connection terminal 311 provides electrical connection to the housing 11 for use as the indifferent electrode during unipolar stimulation or sensing. The connection terminals 319, 313, and 315 provide electrical connection to coil electrodes 20, 8 and 23 respectively. Each of these connection terminals 311, 319, 313, and 315 are coupled to the high voltage output circuit 234 to facilitate the delivery of high energy shocking pulses to the heart using one or more of the coil electrodes 8, 20, and 23 and optionally the housing 11.

The connection terminals 317 and 321 provide electrical connection to the helix electrode 17 and the ring electrode 21 positioned in the right atrium. The connection terminals 317 and 321 are further coupled to an atrial sense amplifier 204 for sensing atrial signals such as P-waves. The connection terminals 323 and 325 provide electrical connection to the helix electrode 26 and the ring electrode 24 positioned in the right ventricle. The connection terminals 323 and 325 are further coupled to a ventricular sense amplifier 200 for sensing ventricular signals.

The atrial sense amplifier 204 and the ventricular sense amplifier 200 preferably take the form of automatic threshold or gain controlled amplifiers with adjustable sensitivity. The general operation of the ventricular sense amplifier 200 and the atrial sense amplifier 204 may correspond to that disclosed in U.S. Pat. No. 5,117,824, by Keimel, et al., incorporated herein by reference in its entirety. Whenever a signal received by atrial sense amplifier 204 exceeds an atrial sensitivity, a signal is generated on the P-out signal line 206. Whenever a signal received by the ventricular sense amplifier 200 exceeds a ventricular sensitivity, a signal is generated on the R-out signal line 202.

Switch matrix 208 is used to select which of the available electrodes are coupled to a wide band amplifier 210 for use in digital signal analysis. Selection of the electrodes is controlled by the microprocessor 224 via data/address bus 218. The selected electrode configuration may be varied as desired for the various sensing, pacing, cardioversion and defibrillation functions of the device 10.

Signals from the electrodes selected for coupling to bandpass amplifier 210 are provided to multiplexer 220, and thereafter converted to multi-bit digital signals by A/D converter 222, for storage in random access memory 226 under control of direct memory access circuit 228. Microprocessor 224 may employ digital signal analysis techniques to characterize the digitized signals stored in random access memory 226 to recognize and classify the patient's heart rhythm employing any of the numerous signal processing methodologies known in the art. An exemplary tachyarrhythmia recognition system is described in U.S. Pat. No. 5,545,186 issued to Olson et al, incorporated herein by reference in its entirety.

The telemetry circuit 331 receives downlink telemetry from and sends uplink telemetry to an external programmer, as is conventional in implantable anti-arrhythmia devices, by means of an antenna 333. Data to be uplinked to the programmer and control signals for the telemetry circuit are provided by microprocessor 224 via address/data bus 218. EGM data that has been stored upon arrhythmia detection or as triggered by other monitoring algorithms may be uplinked to an external programmer using telemetry circuit 331. Received telemetry is provided to microprocessor 224 via multiplexer 220. Numerous types of telemetry systems known in the art for use in implantable devices may be used.

The remainder of the circuitry illustrated in FIG. 2 is an exemplary embodiment of circuitry dedicated to providing cardiac pacing, cardioversion and defibrillation therapies. The pacer timing and control circuitry 212 includes programmable digital counters which control the basic time intervals associated with various single, dual or multi-chamber pacing modes or anti-tachycardia pacing therapies delivered in the atria or ventricles. Pacer circuitry 212 also determines the amplitude of the cardiac pacing pulses under the control of microprocessor 224.

During pacing, escape interval counters within pacer timing and control circuitry 212 are reset upon sensing of R-waves or P-waves as indicated by signals on lines 202 and 206, respectively. In accordance with the selected mode of pacing, pacing pulses are generated by atrial pacer output circuit 214 and ventricular pacer output circuit 216. The pacer output circuits 214 and 216 are coupled to the desired electrodes for pacing via switch matrix 208. The escape interval counters are reset upon generation of pacing pulses, and thereby control the basic timing of cardiac pacing functions, including anti-tachycardia pacing.

The durations of the escape intervals are determined by microprocessor 224 via data/address bus 218. The value of the count present in the escape interval counters when reset by sensed R-waves or P-waves can be used to measure R—R intervals and P—P intervals for detecting the occurrence of a variety of arrhythmias.

The microprocessor 224 includes associated read-only memory (ROM) in which stored programs controlling the operation of the microprocessor 224 reside. A portion of the random access memory (RAM) 226 may be configured as a number of recirculating buffers capable of holding a series of measured intervals for analysis by the microprocessor 224 for predicting or diagnosing an arrhythmia.

In response to the detection of tachycardia, anti-tachycardia pacing therapy can be delivered by loading a regimen from microprocessor 224 into the pacer timing and control circuitry 212 according to the type of tachycardia detected. Alternatively, circuitry for controlling the timing and generation of anti-tachycardia pacing pulses as generally described in U.S. Pat. No. 4,577,633 issued to Berkovits et al., U.S. Pat. No. 4,880,005 issued to Pless et al., U.S. Pat. No. 4,726,380 issued to Vollmann et al., and U.S. Pat. No. 4,587,970 issued to Holley et al., all of which patents are incorporated herein by reference in their entireties, may be used.

In the event that higher voltage cardioversion or defibrillation pulses are required, microprocessor 224 activates the cardioversion and defibrillation control circuitry 230 to initiate charging of the high voltage capacitors 246 and 248 via charging circuit 236 under the control of high voltage charging control line 240. The voltage on the high voltage capacitors is monitored via a voltage capacitor (VCAP) line 244, which is passed through the multiplexer 220. When the voltage reaches a predetermined value set by microprocessor 224, a logic signal is generated on the capacitor full (CF) line 254, terminating charging. The defibrillation or cardioversion pulse is delivered to the heart under the control of the pacer timing and control circuitry 212 by an output circuit 234 via a control bus 238. The output circuit 234 determines the electrodes used for delivering the cardioversion or defibrillation pulse and the pulse wave shape.

One embodiment of an appropriate system for delivery and synchronization of ventricular cardioversion and defibrillation pulses and for controlling the timing function related to them is generally disclosed in commonly assigned U.S. Pat. No. 5,188,105 to Keimel, incorporated herein by reference in its entirety. If atrial defibrillation capabilities are included in the device, appropriate systems for delivery and synchronization of atrial cardioversion and defibrillation pulses and for controlling the timing function related to them may be found in U.S. Pat. No. 4,316,472 issued to Mirowski et al., U.S. Pat. No. 5,411,524 issued to Mehra, or U.S. Pat. No. 6,091,988 issued to Warman, all of which patents are incorporated herein by reference in their entireties. Any known ventricular cardioversion or defibrillation pulse control circuitry may be usable in conjunction with the present invention. For example, circuitry controlling the timing and generation of cardioversion and defibrillation pulses as disclosed in U.S. Pat. No. 4,384,585, issued to Zipes, U.S. Pat. No. 4,949,719, issued to Pless et al., and in U.S. Pat. No. 4,375,817, issued to Engle et al., all incorporated herein by reference in their entireties may be used in a device employing the present invention.

In the illustrated device, delivery of cardioversion or defibrillation pulses is accomplished by output circuit 234, under control of control circuitry 230 via control bus 238. Output circuit 234 determines the shock pulse waveform, e.g. whether a monophasic, biphasic or multiphasic pulse is delivered, whether the housing 311 serves as cathode or anode, which electrodes are involved in delivery of the pulse, and the pulse shape and tilt. Examples of high-voltage cardioversion or defibrillation output circuitry are generally disclosed in U.S. Pat. No. 4,727,877 issued to Kallok, and U.S. Pat. No. 5,163,427 issued to Keimel, both incorporated herein by reference in their entirety.

Examples of output circuitry for delivery of biphasic pulse regimens may be found in U.S. Pat. No. 5,261,400 issued to Bardy, and U.S. Pat. No. 4,953,551 issued to Mehra et al., incorporated herein by reference in its entirety. An example of circuitry which may be used to control delivery of monophasic pulses is set forth in the above cited U.S. Pat. No. 5,163,427, to Keimel. However, output control circuitry for generating a multiphasic defibrillation pulse as generally disclosed in U.S. Pat. No. 4,800,883, issued to Winstrom, incorporated herein by reference in its entirety, may also be used in conjunction with a device embodying the present invention.

In modern implantable cardioverter defibrillators, the particular therapies are programmed into the device ahead of time by the physician, and a menu of therapies is typically provided. For example, on initial detection of tachycardia, an anti-tachycardia pacing therapy may be selected. On redetection of tachycardia, a more aggressive anti-tachycardia pacing therapy may be scheduled. If repeated attempts at anti-tachycardia pacing therapies fail, a higher-level cardioversion pulse therapy may be selected thereafter. As in the case of currently available implantable cardioverter defibrillators (ICDs), and as discussed in the above-cited references, the amplitude of the defibrillation shock may be incremented in response to failure of an initial shock or shocks to terminate fibrillation. Prior art patents illustrating such pre-set therapy menus of anti-tachycardia therapies include the above-cited U.S. Pat. No. 4,726,380 issued to Vollmann et al., above cited U.S. Pat. No. 4,587,970 issued to Holley et al., and U.S. Pat. No. 4,830,006 issued to Haluska, incorporated herein by reference in their entirety.

For purposes of illustrating the invention, known tachyarrhythmia detection methodologies may be utilized, including detection methods as described in U.S. Pat. No. 5,991,656, issued to Olson, et al. on Nov. 23, 1999, U.S. Pat. No. 5,755,736, issued to Gillberg, et al. on May 26, 1998, both incorporated herein by reference in their entireties, or other known ventricular and/or atrial tachyarrhythmia detection methods may be substituted.

It is believed that the method for controlling delivery of pacing pulses of the present invention may be usefully practiced in conjunction with virtually any underlying atrial or ventricular tachyarrhythmia detection scheme. Other exemplary detection schemes are described in U.S. Pat. No. 4,726,380, issued to Vollmann, U.S. Pat. No. 4,880,005, issued to PIess et al., U.S. Pat. No. 4,830,006, issued to Haluska et al., and U.S. patent application Ser. No. 09/566,477, filed May 8, 2000 by Gillberg et al., all incorporated by reference in their entireties herein. However, other criteria may also be measured and employed in conjunction with the present invention.

Criteria for detecting premature contractions may also be event interval based. For example, premature ventricular contractions (PVCs) may be based on the detection of two ventricular events in a row without an intervening atrial event. Detection of runs of premature atrial contractions (PACs) may be based on sensing alternating short and long P—P intervals while isolated PACs may be detected when two successive atrial events are sensed without an intervening ventricular event or when a measured P—P interval is less than a running median or mean P—P interval.

For purposes of the present invention, the particular details of implementation of the rate/interval based arrhythmia detection methodologies are not of primary importance. However, in applications for controlling delivery of pacing pulses for arrhythmia prevention, it is required that the rate based detection methodologies employed by the device allow identification and detection of rhythms representing an arrhythmia, which may include premature beats. According to one embodiment of the present invention, the number and type of arrhythmia detections made during application of controlling delivery of pacing pulses will be used in determining an optimal pacing rate for arrhythmia prevention, as will be described in greater detail below.

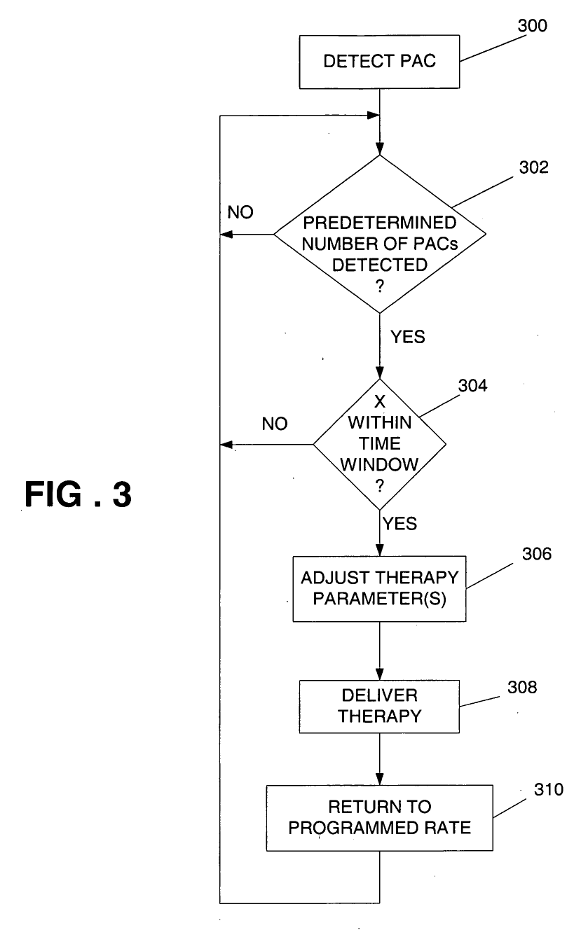

FIG. 3 is a flowchart of a method for controlling delivery of a therapy in an implantable medical device according to an embodiment of the present invention. As illustrated in FIG. 3, premature atrial contractions (PACs) are identified using known methods for identifying PACs, including those described above, Block 300. For example, according to an embodiment of the present invention, a PAC is defined as corresponding to a PP interval less than 75% of a current twelve beat PP interval median.

Once a predetermined number of PACs have been identified, YES in Block 302, a determination is made as to whether the predetermined number of identified PACs occurred within a predetermined time window corresponding to an increase in the frequency of occurrences of PACs that may be indicative of the onset of an atrial arrhythmia, Block 304. For example, according to one embodiment of the present invention, the predetermined number of PACs necessary in Block 302 to trigger the frequency determination in Block 304 is set as three, with the associated time window associated with the frequency determination of Block 304 being set at 30 seconds. As a result, an increase in the frequency of PACs that is indicative of the onset of an atrial tachyarrhythmia would be determined to occur once three PACs are detected within a thirty second time window.

It is understood that the values chosen for the predetermined number of PACs and for the time window are programmable and could be set equal to values other than three PACs being detected within a thirty second time window. For example, according to an embodiment of the present invention, the predetermined number of PACs necessary in Block 302 to trigger the frequency determination in Block 304 is set as five, with the associated time window associated with the frequency determination of Block 304 being set at 45 seconds, so that an increase in the frequency of PACs indicative of the onset of an atrial tachyarrhythmia would be determined to occur once five PACs are detected within a 45 second time window.

Once the predetermined number of PACs are detected within the time window, YES in Blocks 302 and 304, parameters, such as the overdrive pacing rate and the duration of the delivery of the overdrive pacing are adjusted, Block 306. For example, according to an embodiment of the present invention, the pacing rate of the device is adjusted from the nominal programmed rate to an overdrive pacing rate and the duration of the delivery of the overdrive pacing is adjusted from the programmed nominal duration for delivering pacing pulses to an adjusted duration associated with overdrive pacing. Once the parameters have been adjusted, the overdrive pacing therapy is applied at the adjusted rate for a predetermined period of time corresponding to the adjusted duration, Block 308.

It is understood that the monitoring of the frequency of PACs indicative of the onset of an atrial arrhythmia according to the present invention occurs outside the state of arrhythmia detection. Once an arrhythmia is detected, either prior to delivery of the overdrive pacing therapy or during delivery of the overdrive pacing therapy, delivery of the overdrive pacing therapy, the PAC detection, and the monitoring of the frequency of PACs is suspended until the arrhythmia has terminated. Therefore, the overdrive pacing therapy is applied at the adjusted rate for the predetermined period of time corresponding to the adjusted duration in Block 308 or until an arrhythmia is detected by the device, which ever occurs first.

The values for the overdrive pacing rate and the predetermined time period for which the pacing therapy is delivered at the adjusted rate are programmable and could include any desired value. For example, according to one embodiment of the present invention, the overdrive pacing rate is adjusted to 80 beats per minute and the predetermined time period is set as ten minutes, so that the overdrive pacing therapy is delivered in Block 308 at 80 beats per minute for ten minutes. According to another embodiment of the present invention, the overdrive pacing rate is adjusted to 100 beats per minute and the predetermined time period is set as five minutes, so that the overdrive pacing therapy is delivered in Block 308 at 100 beats per minute for five minutes.

Once delivery of the therapy at the adjusted overdrive pacing rate for the predetermined period of time is completed, the pacing rate returns to the initially programmed rate, Block 310, and the detection of increased PAC frequency, Blocks 302 and 304 is repeated. In this way, the present invention adjusts the programmed pacing rate to a fixed overdrive pacing rate in response to an increase in the frequency of PACs and maintains that rate throughout delivery of the overdrive pacing so that the overdrive pacing is delivered at a constant, pre-specified rate. In addition, by deploying the overdrive pacing therapy only in the presence of an increased frequency of PACs, rather than responding to isolated PACs, the present invention is more effective at preventing arrhythmias and reduces the frequency of changes in the pacing rate, making the pacing therapy more tolerable to the patient. Alternately, if an arrhythmia is detected during the therapy period, the therapy is aborted and the device reverts to a normal operating made.

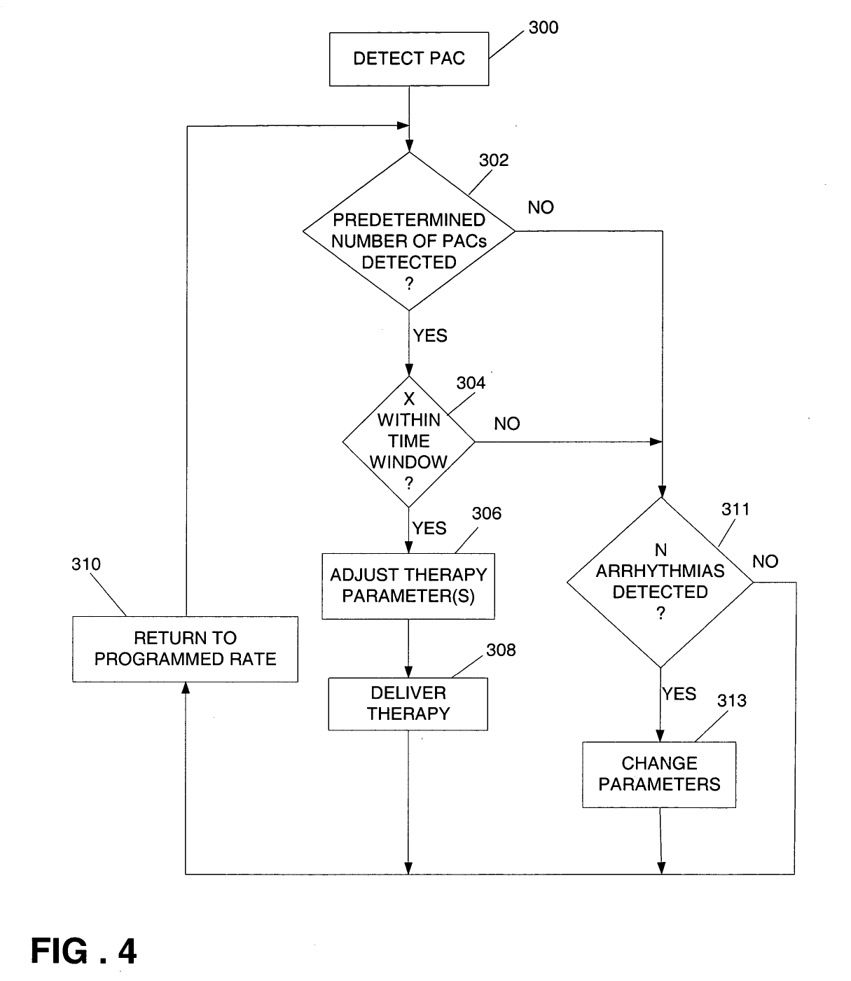

FIG. 4 is a flowchart of a method for controlling delivery of a therapy in an implantable medical device according to an embodiment of the present invention. As illustrated in FIG. 4, according to an embodiment of the present invention, once a predetermined number of PACs have been identified, YES in Block 302, a determination is made as to whether the predetermined number of identified PACs occurred within a predetermined time window corresponding to an increase in the frequency of occurrences of PACs that is indicative of the onset of atrial tachyarrhythmia, Block 304, similar to the embodiment described in FIG. 3. For example, according to one embodiment of the present invention, the predetermined number of PACs necessary in Block 302 to trigger the frequency determination in Block 304 is set as three, with the associated time window associated with the frequency determination of Block 304 being set at 30 seconds. As a result, an increase in the frequency of PACs that is indicative of the onset of atrial tachyarrhythmia would be determined to occur once three PACs are detected within a thirty second time window.

It is understood that the values chosen for the predetermined number of PACs and for the time window are programmable and could be set equal to values other than three PACs being detected within a thirty second time window. For example, according to an embodiment of the present invention, the predetermined number of PACs necessary in Block 302 to trigger the frequency determination in Block 304 is set as five, with the associated time window associated with the frequency determination of Block 304 being set at 45 seconds, so that an increase in the frequency of PACs indicative of the onset of atrial tachyarrhythmia would be determined to occur once five PACs are detected within a 45 second time window.

Once the predetermined number of PACs are detected within the time window, YES in Blocks 302 and 304, parameters, such as the overdrive pacing rate and the duration of the delivery of the overdrive pacing are adjusted, Block 306. For example, according to an embodiment of the present invention, the pacing rate of the device is adjusted from the programmed rate to an overdrive pacing rate and the duration of the delivery of the overdrive pacing is adjusted from the programmed duration for delivering pacing pulses to an adjusted duration. Once the parameters have been adjusted, the overdrive pacing therapy is applied at the adjusted rate for a predetermined period of time corresponding to the adjusted duration, Block 308.

The values for the overdrive pacing rate and the predetermined time period for which the pacing therapy is delivered at the adjusted rate are programmable and could include any desired value. For example, according to one embodiment of the present invention, the overdrive pacing rate is adjusted to 80 beats per minute and the predetermined time period is set as ten minutes, so that the overdrive pacing therapy is delivered in Block 308 at 80 beats per minute for ten minutes. According to another embodiment of the present invention, the overdrive pacing rate is adjusted to 100 beats per minute and the predetermined time period is set as five minutes, so that the overdrive pacing therapy is delivered in Block 308 at 100 beats per minute for five minutes.

Once delivery of the therapy at the adjusted overdrive pacing rate for the predetermined period of time is completed, the pacing rate returns to the initially programmed rate, Block 310, and the detection of increased PAC frequency, Blocks 302 and 304 is repeated. According to the embodiment of FIG. 4, if the predetermined number of PACs have not been detected in Block 302, or if the predetermined number of PACs has been detected but not within the predetermined time window corresponding to an increase in the frequency of occurrences of PACs indicative of onset of an arrhythmia, NO in Block 304, a determination is made as to whether a predetermined number of tachyarrhythmias have occurred for which the overdrive pacing therapy was not delivered, Block 311. If the predetermined number of arrhythmias have occurred for which the overdrive pacing therapy was not delivered, the parameters for detecting an increase in the frequency of PACs, Blocks 302 and 304, are adjusted, Block 313. For example, either the predetermined number of PACs utilized in Block 302 is decreased, or the time window in Block 302 is increased. If the predetermined number of arrhythmias have not occurred, NO in Block 311, or once the parameters have been adjusted in Block 313, the pacing rate returns to the initially programmed rate, Block 310, and the detection of increased PAC frequency, Blocks 302 and 304 is repeated.

In this way, the present invention is able to adjust the sensitivity for determining increased frequency of PACs when multiple arrhythmias are being detecting without deployment overdrive pacing therapy by either reducing the number of beats required to define an increase frequency of PACs and/or by increasing the time window within which the predetermined number of beats must occur.

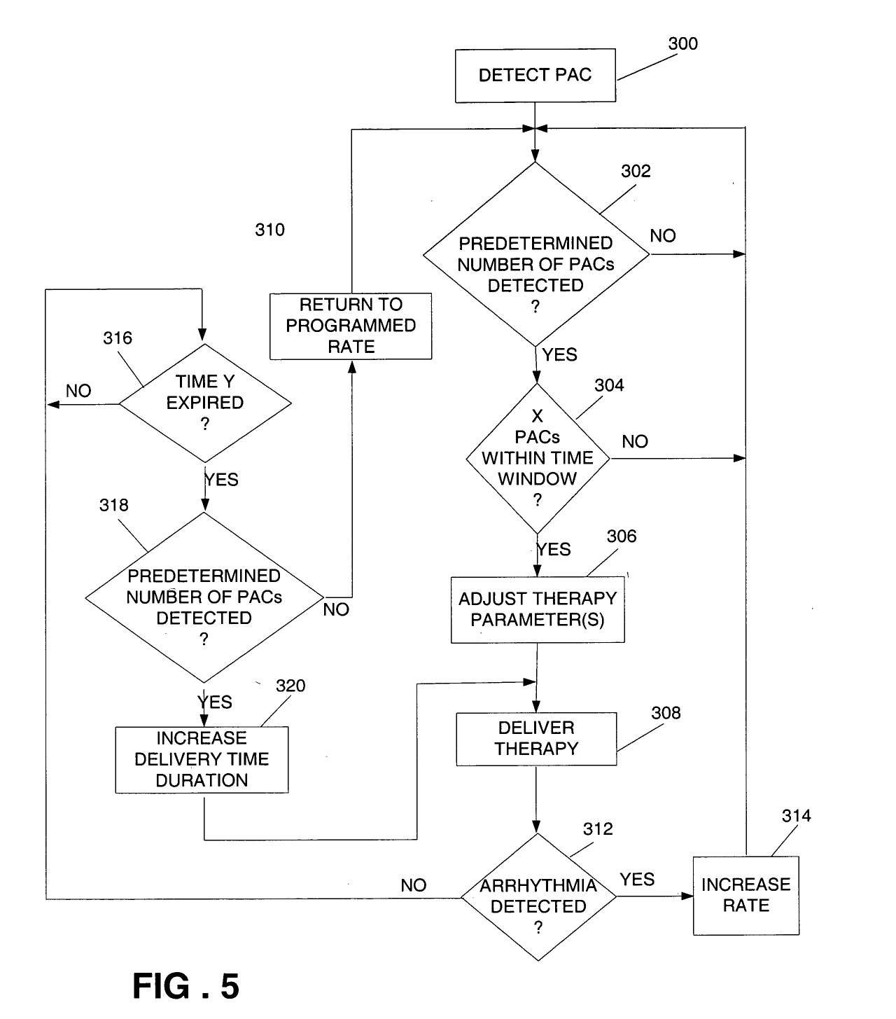

FIG. 5 is a flowchart of a method for controlling delivery of a therapy in an implantable medical device according to an embodiment of the present invention. As illustrated in FIG. 5, additional modifications may be made to the method for controlling delivery of therapy according to the present invention described above. For example, once the predetermined number of PACs have been detected within the predetermined period of time and delivery of the adjusted therapy is completed, Blocks 300-308, a determination is made as to whether an arrhythmia is detected, Block 312. If an arrhythmia is detected once delivery of the overdrive pacing therapy in Block 308 is completed, overdrive pacing therapy is aborted, the device returns to normal operation, and the overdrive pacing rate is increased by a predetermined amount, Block 314 so that the overdrive pacing therapy is subsequently delivered at the increased adjusted rate for the predetermined period of time, Block 308, at the next deployment of overdrive pacing therapy subsequent to detection of an increased PAC frequency, Blocks 302-304.

According to the present invention, the amount that the overdrive pacing rate is increased in Block 314 is programmable and could include any desired increment. For example, according to an embodiment of the present invention, the overdrive pacing rate is increased by a fixed amount, such as by five beats per minute, i.e., from 80 beats per minute to 85 beats per minute, or by a percentage of the current overdrive pacing rate, such as 10%, i.e., from 80 beats per minute to 88 beats per minute. In this way, if the overdrive pacing therapy is not successful in preempting the arrhythmia, the set overdrive pacing rate is increased.

If an arrhythmia is not detected after the overdrive pacing therapy is delivered, NO in Block 312, a determination is made as to whether a predetermined post-therapy time Y has expired since the delivery of the most recent session of overdrive pacing therapy, Block 316. Once the post-therapy time Y has expired, a determination is made as to whether a predetermined number of PACs were detected during the post-therapy time Y, Block 318. If the predetermined number of PACs are detected within the post-therapy time Y, the time duration associated with the delivery of the overdrive pacing therapy is increased, Block 320, and delivery of the overdrive pacing therapy is repeated using the previous overdrive pacing rate for the increased duration or time window, Block 308. Once the subsequent overdrive pacing therapy is delivered at the previous overdrive pacing rate for the increased period of time, the determination as to whether an atrial tachycardia is detected, Block 312, is repeated.

If the predetermined number of PACs have not been detected within the post-therapy time Y, NO in Block 318, the pacing rate returns to the initially programmed rate, Block 310, and the detection of increased PAC frequency, Blocks 302 and 304 is repeated.

According to the present invention, any desired value can be utilized for the post-therapy time Y and the predetermined number of PACs in Blocks 316 and 318, respectively. For example, according to one embodiment of the present invention, time Y is set equal to the time window utilized in Block 304, i.e., 30 seconds, for example, and the number of PACs is set equal to the predetermined number X utilized in Block 302, i.e., 3, so that even though the arrhythmia has been prevented from occurring as a result of the delivery of the overdrive pacing therapy, if the frequency of the PACs nonetheless continues at an accelerated rate after the initial therapy session is completed, delivery of the overdrive pacing therapy is repeated using an increased duration. For example, according to an embodiment of the present invention, assuming the parameters of the overdrive pacing therapy are initially set at 80 beats per minute for ten minutes, the duration would be increased from ten minutes to fifteen minutes in Block 320 so that the overdrive pacing therapy is subsequently delivered at 80 beats per minute for fifteen minutes in Block 308. While the duration is described as being increased from ten minutes to fifteen minutes, the duration could be increased by any desired amount without departing from the present invention.

Alternately, if the frequency of the PACs nonetheless continues at an accelerated rate after the initial therapy session is completed, delivery of the overdrive pacing therapy is repeated using an increased duration and/or an increased overdrive pacing rate.

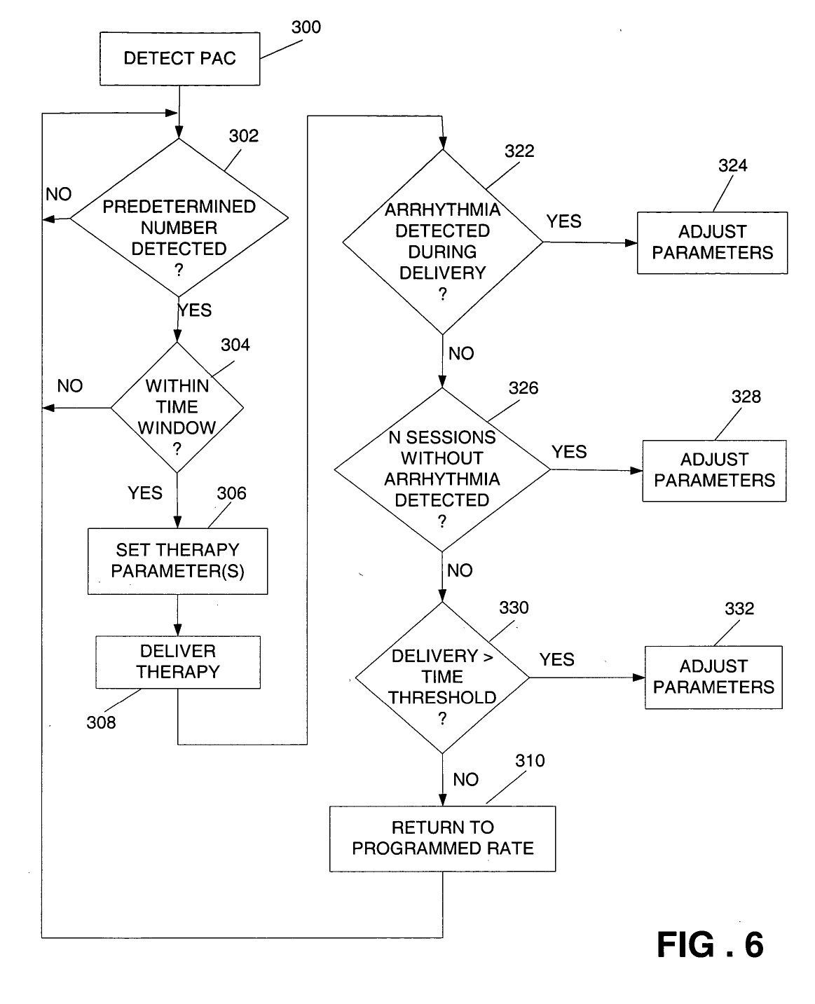

FIG. 6 is a flowchart of a method for controlling delivery of a therapy in an implantable medical device according to an embodiment of the present invention. As illustrated in FIG. 6, further additional modifications may be made to the method for controlling delivery of therapy according to the present invention described above. For example, according to an embodiment of the present invention, once the predetermined number of PACs have been detected within the predetermined period of time and delivery of the adjusted therapy is completed, Blocks 300-308, a determination is made as to whether a tachyarrhythmia, such as an atrial tachycardia for example, was detected during delivery of the overdrive pacing therapy, Block 322. If an atrial tachycardia was detected during therapy delivery, the therapy delivery parameters are adjusted, Block 324. For example, in one embodiment, the overdrive pacing rate is increased by a predetermined amount. The amount that the overdrive pacing rate is increased in Block 314 is programmable and could include any desired increment. For example, the overdrive pacing rate can be increased by a fixed amount, such as by five beats per minute, i.e., from 80 beats per minute to 85 beats per minute, or by a percentage of the current overdrive pacing rate, such as 10%, i.e., from 80 beats per minute to 88 beats per minute.

It is understood that the present invention is not intended to be limited to increasing the overdrive pacing rate in response to an arrhythmia being detected during delivery of the overdrive pacing therapy. For example, according to an embodiment of the present invention, the duration of the delivery of the overdrive pacing therapy could be increased, or both the delivery rate and delivery duration of the therapy could be increased.

According to another embodiment of the present invention, if an arrhythmia is detected in Block 322, such information is stored in memory 226 and subsequently downloaded from the device to a programmer, such as, for example, during a subsequent office visit or other interrogation by the patient. As a result, a physician or clinician who is made aware that an arrhythmia was detected during delivery of the overdrive pacing therapy can then increase the overdrive pacing rate during the next office visit, rather than being increased automatically by the device, as described above. According to another embodiment of the present invention, either the pacing rate or the duration or both may be increased.

According to an embodiment of the present invention, once the pacing rate or other parameters have been adjusted in Block 324, or if an arrhythmia is not detecting during delivery of the overdrive pacing therapy, NO in Block 322, a determination is made as to whether a predetermined number N of overdrive pacing therapy sessions have been delivered without an arrhythmia being detected, Block 326. For example, according to one embodiment of the present invention, a determination is made in Block 326 as to whether, for four out of five overdrive pacing therapy sessions that have been delivered, no arrhythmia was detected. It is understood that although four out of five overdrive pacing therapy sessions is described to determine the effectiveness of the algorithm, any numbers of sessions may be utilized without departing from the present invention.

If the predetermined number N of overdrive pacing therapy sessions have been delivered without detecting an arrhythmia, the parameters for detecting an increased PAC frequency, Blocks 302 and 304, are adjusted, Block 328, in order to reduce the triggering criteria for determining that there is an increase in the frequency of PACs. For example, either the predetermined number of PACs utilized in Block 302 is increased, or the time window utilized in Block 304 is decreased. According to an embodiment of the present invention, if the predetermined number N of overdrive pacing therapy sessions have been delivered without detecting an arrhythmia, the overdrive pacing rate and/or the overdrive pacing duration could be reduced, rather than or in addition to the PAC frequency parameters.

Once the parameters for detecting an increased PAC frequency have been adjusted in Block 328 to a greater number PACs or a shorter duration window, or if the predetermined number N of overdrive pacing therapy sessions have not been completed without detecting an arrhythmia, NO in Block 326, a determination is made as to whether the overdrive pacing therapy has been delivered in Block 308 more than a predetermined time threshold, Block 330. For example, according to an embodiment of the present invention, a determination is made in Block 330 as to whether the overdrive pacing therapy has been delivered a certain percentage of a given time period, such as 80% of the time over the last 24 hours. However, the threshold of Block 330 is programmable and therefore may set at any desired threshold value.

If the overdrive pacing therapy has been delivered for a period of time greater than the time threshold, the parameters for detecting an increased PAC frequency, Blocks 302 and 304, are adjusted, Block 332, in order to reduce the frequency of delivery of the overdrive pacing in response to increased frequency of PACs. For example, either the predetermined number X of PACs utilized in Block 302 is increased, or the time window in Block 304 is decreased. Once the parameters for detecting an increased PAC frequency have been adjusted in Block 332, or if the overdrive pacing therapy has not been delivered more than the time threshold, NO in Block 330, the pacing rate returns to the initially programmed rate, Block 310, and the detection of increased PAC frequency, Blocks 302 and 304 is repeated.

As described above, the present invention automatically adjusts the programmed pacing rate to a fixed overdrive pacing rate in response to the device classified recurrence rate of PACs or arrhythmias to further enhance the performance of the method for controlling delivery of a therapy in an implantable medical device according to an embodiment of the present invention. It is understood that while the flowchart of FIG. 6 includes examples of certain adjustments of operation of the device in response to the device classified recurrence rate of PACs or arrhythmias, i.e., Blocks 322 and 324, 326 and 328, and 330 and 332, the present invention is not intended to be limited to a device that includes the combination of automatic adjustments described. Rather, the present invention may include any single adjustment or combination of adjustments, including the adjustment described above in Blocks 312-320 of FIG. 5 and made either automatically by the device, or manually by a clinician upon retrieval of the adjustment determinations from memory.

It is understood that while the determination of whether there is an increased frequency of PACs indicative of the onset of an atrial arrhythmia is described above as being a determination of the number of PACs detected within a time window, other methods may be utilized for detecting an increase in PAC frequency without departing from the scope of the present invention. For example, according to an embodiment of the present invention, an increase in PAC frequency is determined to occur when the coupling interval of a most recent detected PAC is shorter than the coupling interval associated with a previous detected PAC, or shorter than an average of previously detected PACs, and so forth.

Some of the techniques described above may be embodied as a computer-readable medium comprising instructions for a programmable processor such as microprocessor 224 or control circuitry 212 shown in FIG. 2. The programmable processor may include one or more individual processors, which may act independently or in concert. A “computer-readable medium” includes but is not limited to any type of computer memory such as floppy disks, conventional hard disks, CR—ROMS, Flash ROMS, nonvolatile ROMS, RAM and a magnetic or optical storage medium. The medium may include instructions for causing a processor to perform any of the features described above for initiating a session of the escape rate variation according to the present invention.

The preceding specific embodiments are illustrative of the practice of the invention. It is to be understood, therefore, that other expedients known to those of skill in the art or disclosed herein may be employed without departing from the invention or the scope of the appended claim. It is therefore to be understood that the invention may be practiced otherwise than as specifically described, without departing from the scope of the present invention. As to every element, it may be replaced by any one of infinite equivalent alternatives, only some of which are disclosed in the specification.

1. An implantable medical device, comprising:
means for sensing a plurality of events;
means for detecting whether there is an increase in the frequency of first events of the plurality of events corresponding to onset of a second event of the plurality of sensed events;
means for adjusting parameters associated with delivery of the therapy in response to the detected increased frequency of first sensed events; and
means for delivering the therapy using the adjusted parameters.

2. The device of claim 1, wherein the first events correspond to premature atrial contractions and the means for detecting whether there is an increase in the frequency of first events determines whether a predetermined number of premature atrial contractions occur within a predetermined time window.

3. The device of claim 1, wherein the means for adjusting parameters associated with delivery of the therapy adjusts one of a rate of delivery of the therapy and a duration of delivery of the therapy at the adjusted rate.

4. The device of claim 1, further comprising:
means for determining, in response to an increase in the frequency of the first events not being detected, whether a predetermined number of the second event have occurred for which the therapy using the adjusted parameters has not been delivered; and
means for adjusting parameters associated with detecting whether there is an increase in the frequency of first events in response to the predetermined number of the second event not occurring.

5. The device of claim 4, wherein the first events correspond to premature atrial contractions and the means for detecting whether there is an increase in the frequency of first events determines whether a predetermined number of premature atrial contractions occur within a predetermined time window, and wherein the means for adjusting parameters associated with detecting an increase in the frequency of first sensed events adjusts one of the predetermined number of premature atrial contractions and the predetermined time window.

6. The device of claim 3, further comprising:
means for determining whether the second event is detected subsequent to delivery of the therapy; and
means for increasing the delivery rate of the therapy in response to the second event being detected.

7. The device of claim 3, further comprising:
means for determining whether the second event is detected subsequent to delivery of the therapy;
means for determining whether a predetermined number of the first event occur within a predetermined time period subsequent to delivery of the therapy; and
means for increasing one of the delivery duration and the delivery rate in response to the predetermined number of the first event occurring with the predetermined time period.

8. The device of claim 3, further comprising:
means for determining whether the second event is detected during delivery of the therapy; and
means for increasing one of the delivery duration and the delivery rate in response to the second event being detected during delivery of the therapy.

9. The device of claim 2, further comprising:
means for determining whether the therapy has been delivered a predetermined number of times;
means for determining whether the second event was detected subsequent to the delivery of the therapy; and
means for adjusting one of the number of premature atrial contractions and the time window in response to the second event not being detected subsequent to the delivery of the therapy.

10. The device of claim 3, further comprising:
means for determining whether the therapy has been delivered a predetermined number of times;
means for determining whether the second event was detected subsequent to the delivery of the therapy; and
means for adjusting one of the delivery duration and the delivery rate in response to the second event not being detected subsequent to the delivery of the therapy.

11. The device of claim 2, further comprising:
means for determining whether the therapy has been delivered more than a predetermined time threshold; and
means for adjusting one of the number of premature atrial contractions and the time window in response to the therapy being delivered more than the predetermined time threshold.

12. A method of controlling delivery of therapy in an implantable medical device, comprising:
sensing a plurality of events;
detecting whether there is an increase in the frequency of first events of the plurality of events corresponding to onset of a second event of the plurality of sensed events;
adjusting parameters associated with delivery of the therapy in response to the detected increased frequency of first sensed events; and
delivering the therapy using the adjusted parameters.

13. The method of claim 12, wherein the first events correspond to premature atrial contractions and detecting whether there is an increase in the frequency of first events comprises determining whether a predetermined number of premature atrial contractions occur within a predetermined time window.

14. The method of claim 12, wherein adjusting parameters associated with delivery of the therapy comprises adjusting one of a rate of delivery of the therapy and a duration of delivery of the therapy at the adjusted rate.

15. The method of claim 12, further comprising:
determining, in response to an increase in the frequency of the first events not being detected, whether a predetermined number of the second event have occurred for which the therapy using the adjusted parameters has not been delivered; and
adjusting parameters associated with detecting whether there is an increase in the frequency of first events in response to the predetermined number of the second event not occurring.

16. The method of claim 15, wherein the first events correspond to premature atrial contractions and detecting whether there is an increase in the frequency of first events comprises determining whether a predetermined number of premature atrial contractions occur within a predetermined time window, and wherein adjusting parameters associated with detecting an increase in the frequency of first sensed events comprises one of adjusting the predetermined number of premature atrial contractions and the predetermined time window.

17. The method of claim 14, further comprising:
determining whether the second event is detected subsequent to delivery of the therapy; and
increasing the delivery rate of the therapy in response to the second event being detected.

18. The method of claim 14, further comprising:
determining whether the second event is detected subsequent to delivery of the therapy;
determining whether a predetermined number of the first event occur within a predetermined time period subsequent to delivery of the therapy; and
increasing one of the delivery duration and the delivery rate in response to the predetermined number of the first event occurring with the predetermined time period.

19. The method of claim 14, further comprising:
determining whether the second event is detected during delivery of the therapy; and
increasing one of the delivery duration and the delivery rate in response to the second event being detected during delivery of the therapy.

20. The method of claim 13 further comprising:
determining whether the therapy has been delivered a predetermined number of times;
determining whether the second event was detected subsequent to the delivery of the therapy; and
adjusting one of the number of premature atrial contractions and the time window in response to the second event not being detected subsequent to the delivery of the therapy.

21. The method of claim 14, further comprising:
determining whether the therapy has been delivered a predetermined number of times;
determining whether the second event was detected subsequent to the delivery of the therapy; and
adjusting one of the delivery duration and the delivery rate in response to the second event not being detected subsequent to the delivery of the therapy.

22. The method of claim 13, further comprising:
determining whether the therapy has been delivered more than a predetermined time threshold; and
adjusting one of the number of premature atrial contractions and the time window in response to the therapy being delivered more than the predetermined time threshold.

23. A computer-readable medium having computer-executable instructions for performing a method, comprising:
means for sensing a plurality of events;
means for detecting whether there is an increase in the frequency of first events of the plurality of events corresponding to onset of a second event of the plurality of sensed events;
means for adjusting parameters associated with delivery of the therapy in response to the detected increased frequency of first sensed events; and
means for delivering the therapy using the adjusted parameters.
